(19) United States
(12) Patent Application Publication
(10) Pub. No.: US 2020/0209238 A1
(43) Pub. Date: Jul. 2, 2020

(54) OPTIMIZING DIAGNOSTICS FOR GALACTOFURANOSE CONTAINING ANTIGENS

(71) Applicant: The Johns Hopkins University, Baltimore, MD (US)

(72) Inventors: Kieren Marr, Baltimore, MD (US); Kausik Datta, Baltimore, MD (US)

(21) Appl. No.: 16/612,372

(22) PCT Filed: May 9, 2018
(86) PCT No.: PCT/US2018/031888

§ 371 Date: Nov. 9, 2019

Related U.S. Application Data
(60) Provisional application No. 62/503,492, filed on May 9, 2017.

(51) Int. Cl. G01N 33/569; C07K 16/28

(52) U.S. Cl. CPC G01N 33/569; G01N 2400/32; C07K 16/2851

(57) ABSTRACT

Disclosed herein are methods of detecting microbial infection in mammalian subjects comprising treatment of a sample and detection of polysaccharide antigenic components. The methods disclosed provide for pretreatment of biological samples, such as urine samples, to maximize detection of galF—containing antigens and improvement of sensitivity of galF antigen detection assays. The methods include minimizing Intelectin-1 binding to galF—containing antigens and improvement of monoclonal antibody binding. The detection methods are useful for identifying the presence of microbial antigens related to Streptococcus pneumoniae, Klebsiella pneumonia, Escherichia coli, Mycobacteria species, Malassezia species, Aspergillus species, Fusarium species, Alternaria species, Coccidioides species, Cryptococcus species, Mucormycetes, Histoplasma species, Neosartorya species, Fusarium species, Paracoccidioides species, or combinations thereof.

### CROSS REFERENCE TO RELATED APPLICATIONS

This application claims the benefit of priority of U.S. Provisional Application 62/503,492 filed on May 9, 2017, the entire contents of which are incorporated herein by reference in its entirety.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Apr. 6, 2017, is named P14185-01_ST25.txt and is 5,759 bytes in size.

### BACKGROUND

Galactose is common in mammals, but only found in the 6-member ring hexopyranosyl form, called galactopyranose (galP). Galactofuranose (galF), the 5-member ring form of galactose, is found in other organisms, including bacteria, fungi, protozoa, lichens, green algae, starfish and sponges. Equilibrium strongly favors the galP form unless the organism contains specific enzymes to catalyze formation of galF. In these organisms, galF is an important residue on glycoconjugate antigens, and can be found linked to secreted and cellular polysaccharides, glycoproteins and glycosphingolipids.

### SUMMARY

In accordance with one or more embodiments, the present invention provides methods for improved galF antigen detection in human body fluids by disabling or otherwise removing a competitive inhibitor, the human lectin, intelectin-1 from an assay process such as the assay previously disclosed in U.S. patent application Ser. No. 13/511,264, and incorporated by reference herein in its entirety. Although intelectin-1 is known to be secreted by epithelial cells lining the GI tract and respiratory tract, and for recognizing galF-containing antigens, intelectin was not known until now to be present in urine. The present inventors surprisingly determined that intelectin-1 is also present in urine, and serves to compete with galF-binding antibodies when they are used as part of diagnosing microbial infections in a mammalian subject. Though not wishing to be bound by the following theory, it is thought that this inhibition is dependent on calcium, which can be removed by chelation as a mechanism to optimize antigen identification.

In some embodiments, the microbial infection is caused by an organism comprising: Streptococcus pneumoniae, Klebsiella pneumonia, Escherichia coli, Mycobacteria species, Malassezia species, Aspergillus species, Fusarium species, Alternaria species, Coccidioides species, Cryptococcus species, Mucormycetes, Histoplasma species, Neosartorya species, Fusarium species, Paracoccidioides species, or combinations thereof.

As such, in accordance with one or more embodiments, the present invention provides methods for optimization of galF-antigen identification in fluids that contain intelectin-1, including urine, respiratory fluids, gastrointestinal fluids, and blood. The present inventors found that this is important for optimizing those methods that specifically focus on fungal antigens. The utility is broadly applicable to diagnostics that target galF containing antigens in many different diagnostic systems, given the ubiquity of galF in microbial antigens.

In accordance with an embodiment, the present invention provides a method for diagnosing a microbial infection in a biological sample from a mammalian subject suspected of having, having, or susceptible to having a microbial infection by detecting the presence of at least one polysaccharide comprising a galactofuranose (galF) residue in a biological sample of the mammalian subject, the method comprising: (a) treating the biological sample to inhibit or interfere with human intelectin-1 (hIntL-1) binding of galF residues present in the sample; (b) allowing the treated sample of (a) to come in contact with at least one antibody specific for at least one polysaccharide comprising a galF residue in an effective amount to produce a detectable amount of antibody-polysaccharide complex; and (c) detecting the presence of at least one antibody-polysaccharide complex, wherein the detection of the presence of at least one antibody-polysaccharide complex is diagnostic of a microbial infection in a mammalian subject. In certain embodiments, the biological sample comprises galactofuranose.

In accordance with another embodiment, the present invention provides a method for diagnosing a microbial infection in a biological sample from a mammalian subject suspected of having, having, or susceptible to having a microbial infection by detecting the presence of at least one polysaccharide comprising a galF residue in a biological sample of the mammalian subject, the method comprising: (a) treating the biological sample comprising contacting the sample with a substrate which binds divalent cations with high affinity, thereby inhibiting hIntL-1 binding of galF residues present in the sample; (b) contacting the treated sample of (a) with at least one antibody specific for at least one polysaccharide comprising a galF residue in an effective amount to produce a detectable amount of antibody-polysaccharide complex; and (c) detecting the presence of at least one antibody-polysaccharide complex, wherein the detection of the presence of at least one antibody-polysaccharide complex is diagnostic of a microbial infection in a mammalian subject.

In accordance with certain embodiments, a method for diagnosing a microbial infection in a biological sample from a mammalian subject suspected of having, having, or susceptible to having a microbial infection, comprises (a) treating the biological sample to inhibit human intelectin-1 (hIntL-1) binding of galactofuranose residues present in the biological sample; (b) contacting the treated sample of (a) with at least one antibody specific for at least one polysaccharide comprising a galactofuranose residue in an effective amount to produce a detectable amount of antibody-polysaccharide complex; and (c) detecting the presence of at least one antibody-polysaccharide complex, wherein the detection of the presence of at least one antibody-polysaccharide complex is diagnostic of a microbial infection in a mammalian subject.

In accordance with certain embodiments, a method for diagnosing a microbial infection in a biological sample from a mammalian subject suspected of having, having, or susceptible to having a microbial infection, comprises (a) treating the biological sample with one or more compounds; (b) contacting the treated biological sample of (a) with at least one antibody specific for a galactofuranose residue, in an effective amount to produce a detectable amount of antibody-polysaccharide complex; and (c) detecting the presence of at least one antibody-polysaccharide complex, wherein the detection of the presence of at least one antibody-polysaccharide complex is diagnostic of a microbial infection in a mammalian subject.

In accordance with certain embodiments, a method of detecting a microorganism in a sample, comprises: (a) contacting the sample with one or more compounds comprising: glycerol, 3-Keto-2-deoxyoctonic acid (KDO), D-glycerol-1-phosphate, D-mannoheptose lactoferrin, saccharides, D-phospho-glycerol-modified glycans, heptoses, D-glycero-D-talo-oct-2-ulosonic acid (KO), 3-deoxy-D-manno-oct-2-ulosonic acid (KDO), sepharose, variants, derivatives or combinations thereof; (b) contacting the sample with an antibody which specifically binds to polysaccharides comprising galactofuranose; (c) detecting the presence of at least one antibody-polysaccharide complex, thereby detecting the microorganism in the sample. In certain embodiments, the one or more compounds are in solution or immobilized on a support or packed in a column. In certain embodiments, the one or more antibodies are in solution or immobilized on a support or packed in a column. In certain embodiments, the support is a dipstick, bead, chip, microwell, filter, resin, membrane, or quantum dot. The sepharose may be coated with e.g. antibodies, ligands, proteins, sugars and the like, the surface may comprise functionalized groups or chemical entities, etc.

In certain embodiments, a method of removing, decreasing the amount of, eliminating or decreasing interference by a human intelectin molecule from a biological sample, comprises contacting the biological sample containing the human intelectin with a one or more compounds comprising a linear carbohydrate or carbohydrate comprising an exocyclic diol under conditions permitting binding of the human intelectin to the linear carbohydrate. The removal, elimination of or decrease in amount of human intelectin in a biological sample, prevents, reduces or inhibits binding of intelectin to galactofuranose. In certain embodiments, the exocyclic diol is exclusive of galactofuranose.

In accordance with some embodiments, a support substrate comprises at least one antibody specific for at least one galactofuranose residue comprise: monoclonal antibody 205 (MAb 205) comprising a variable heavy (VH) domain of SEQ ID NO:1 and a variable light (VL) domain of SEQ ID NO:2; monoclonal antibody 24 (MAb 24) comprising a VH domain of SEQ ID NO:3 and a VL domain of SEQ ID NO:4; monoclonal antibody 686 (MAb 686) comprising a VH domain of SEQ ID NO:5 and a VL domain of SEQ ID NO:6; monoclonal antibody 838 (MAb 838) comprising a VH domain of SEQ ID NO:7 and a VL domain of SEQ ID NO:8; and monoclonal antibody 476 (MAb 476) comprising a VH domain of SEQ ID NO:9 and a VL domain of SEQ ID NO:10, or combinations thereof. In certain embodiments, the support substrate is a dipstick, bead, chip, microwell, filter, resin, gel, gel matrix, membrane, or quantum dot.

In certain embodiments, a composition comprises at least one antibody specific for at least one galactofuranose residue including but not limited to: monoclonal antibody 205 (MAb 205) comprising a variable heavy (VH) domain of SEQ ID NO:1 and a variable light (VL) domain of SEQ ID NO:2; monoclonal antibody 24 (MAb 24) comprising a VH domain of SEQ ID NO:3 and a VL domain of SEQ ID NO:4; monoclonal antibody 686 (MAb 686) comprising a VH domain of SEQ ID NO:5 and a VL domain of SEQ ID NO:6; monoclonal antibody 838 (MAb 838) comprising a VH domain of SEQ ID NO:7 and a VL domain of SEQ ID NO:8; and monoclonal antibody 476 (MAb 476) comprising a VH domain of SEQ ID NO:9 and a VL domain of SEQ ID NO:10, or combinations thereof. In certain embodiments, the composition is a solution.

In accordance with some embodiments, the method for treating the sample in step (a) comprises contacting the sample with a substrate which binds Ca′ ions with high affinity.

In accordance with some embodiments, the method for treating the sample in step (a) comprises contacting the sample with a compound which chelates Ca′ ions with high affinity.

In accordance with some embodiments, the method for treating the sample in step (a) comprises contacting the sample with EDTA and/or EGTA.

In accordance with some embodiments, the method for treating the sample in step (a) comprises contacting the sample with a substrate which binds hIntL-1 with high affinity.

In accordance with some embodiments, the method for treating the sample in step (a) comprises contacting the sample with an antibody specific for hIntL-1.

In accordance with some embodiments, the method for treating the sample in step (a) comprises contacting the sample with one or more compounds which bind hIntL-1 with high affinity, thereby inhibiting or interfering with galF-binding to hIntL-1.

In accordance with some embodiments, the method for treating the sample in step (a) comprises contacting the sample with one or more compounds which bind hINTL-1 with high affinity comprising: glycerol, 3-Keto-2-deoxyoctonic acid; D-glycerol-1-phosphate, D-mannoheptose, lactoferrin, saccharides, D-phospho-glycerol-modified glycans, heptoses, D-glycero-D-talo-oct-2-ulosonic acid (KO), 3-deoxy-D-manno-oct-2-ulosonic acid (KDO), sepharose or combinations thereof.

In accordance with some embodiments, the method for treating the sample in step (a) comprises contacting the sample with one or more compounds which bind hINTL-1 with high affinity containing sepharose.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The terms “determining”, “measuring”, “evaluating”, “detecting”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “high affinity” when used in context of compounds binding to hINTL-1 refers to compounds having a K_{D} of 1×10⁻⁷ M or less, 5×10⁻⁸ M or less, 1×10⁻⁸M or less, 5×10⁻⁹ M or less 1×10⁻⁹ M or less, 1×10⁻¹⁰ or less, 1×10⁻¹¹ or less, 1×10⁻¹² or less, 1×10⁻¹³ or less, 1×10⁻¹⁴, or less or 1×10⁻¹⁵ or less.

As used in this specification and the appended claims, the term “or” is generally employed in its sense including “and/or” unless the content clearly dictates otherwise.

As used herein, the term “removing”, “eliminating”, “decrease in amount” or “decrease interference by intelectin” of human intelectin in a biological sample, prevents, reduces, inhibits or competes out the binding of intelectin to galactofuranose and other ligands. In some embodiments, “decreasing interference by intelectin”, is be achieved by, but not limited to, reducing binding of intelectin to galactofuranose.” The amount of human intelectin in the biological sample can be measured via any assay, such as for example, immunoassays, chromatography, gels, and the like.

The recitation of numerical ranges by endpoints includes all numbers within that range (e.g., 1 to 5 includes 1, 1.5, 2, 2.75, 3, 3.80, 4, and 5). Although some suitable dimensions ranges and/or values pertaining to various components, features and/or specifications are disclosed, one of skill in the art, incited by the present disclosure, would understand desired dimensions, ranges and/or values may deviate from those expressly disclosed.

### BRIEF DESCRIPTION OF THE DRAWINGS

FIG. 1 illustrates the inventors' prior discovery of galF antigens in the urine of patients with Aspergillus infection and production of monoclonal antibodies specific for galF antigens, with sensitivity to detect excreted antigens in urine samples.

FIG. 2 shows intelectin and what is known about lectin molecules. Intelectin was previously called ‘omentin’ as it is secreted from adipose cells and epithelial cells. Intelectin binds to certain glycoproteins, especially those produced by microbes and other non-mammalian organisms (ex. algae, plants). Intelectin-1 is thought to be involved in host-defense as it is frequently found in the lungs and gut surfaces that are facing the environment.

FIG. 3 shows the inventors' hypothesis that INTL-1 was secreted into the urine and was competitively binding to galF antigens in the presence of the inventors' monoclonal antibodies specific for galF, and that desalting removes Ca²⁺ needed for INTL-1 to bind galF.

FIGS. 4A-4B depict (FIG. 4A) rINTL-1 binds to Aspergillus ethanol precipitate (EP) in an ELISA, and (FIG. 4B) binds to whole cells (conidia & hyphae, red). Binding is galF-specific, as the galF-specific monoclonal antibody mAb46 reduces cellular rINTL-1 binding, but irrelevant antibody does not (FIG. 4B) FIG. 4B: Af293 germinating conidia and hypha stained with recombinant hINLT (RED), with concurrent mAb476, a galF-specific antibody, compared to an irrelevant mouse IgM. hINTL binds to galF on the surface of growing hyphae and conidia. GalF recognition is reduced by mAb476.

FIGS. 5A and 5B show the presence of hINTL-1 in urine from a patient with aspergillosis and healthy control (pooled from 4 donors). FIG. 5A: Radiograms from Western blots show bands in healthy urine that migrate at 120 kDa in non-reducing conditions, corresponding to the trimeric form; in reducing conditions, monomeric forms are seen at lower molecular weights. Patient urine contains high amounts of the low molecular weight monomeric forms in both reducing and non-reducing conditions. FIG. 5B: Enzyme immunoassays demonstrate higher concentrations of hINTL in urines from two patients, and measurable amounts in healthy pooled urine.

FIG. 6 shows that galF specific antibody mAB476 binds to the band that co-migrates with that recognized by anti-INTL-1 and shows that when the aspergillosis patient urine is desalted prior to contacting the urine with the antibody, the amount of antibody binding is increased.

FIG. 7 shows schematic of immunoprecipitation experiments performed to identify mAb476 bound antigens in urine from patients with aspergillosis. To enrich sample detection of moieties specifically binding to mAb476, urines were incubated with superparamagnetic dynabeads coated with mAb476 and a mouse control antibody. mAB476 reactive material was eluted with 50 mM glycine (pH 2.5-3.0). No mAB476 binding occurred in normal urine. Reactivity to mAb476 eluates was confirmed by sandwich ELISA. Eluates were then run on SDS-PAGE gels to check for band patterns, and in-solution mass spectrometry analysis was performed. Analysis detected both the microbial antigen CelA/Aspf7 (a galF-containing O-glycan), and the human INTL-1 protein.

FIGS. 8 and 9 shows that treatment of the patient urine with calcium chelators or other compounds which bind calcium or compete with galF for IntL-1 binding with high affinity, when added prior to contacting the sample with galF antibodies, significantly improves detection sensitivity.

FIGS. 10A and 10B show that treatment of the patient urine by passage through a sepharose containing column increases the signal produced by mAb476 binding on a lateral flow dipstick assay (with both control and test lines shown in red) in both healthy urine spiked with Aspergillus ethanol precipitate (EP, FIG. 10A) and in urine from a patient with aspergillosis (FIG. 10B).

FIG. 11 shows that hINTL-1 concentration is decreased in human urine that is treated by exposure to sepharose-containing columns (flow-through).

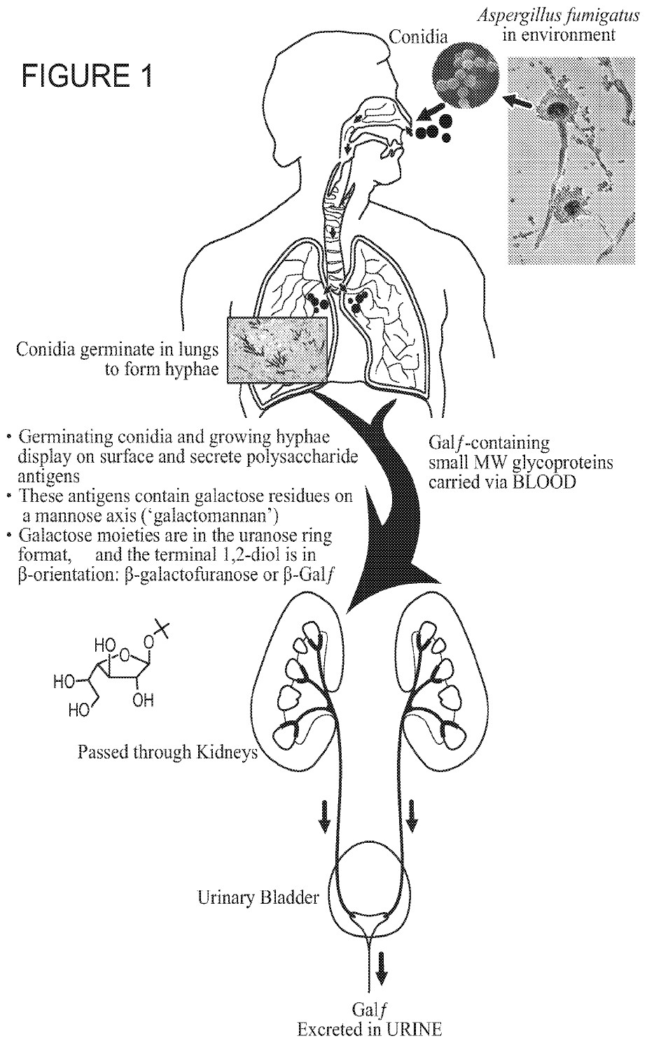

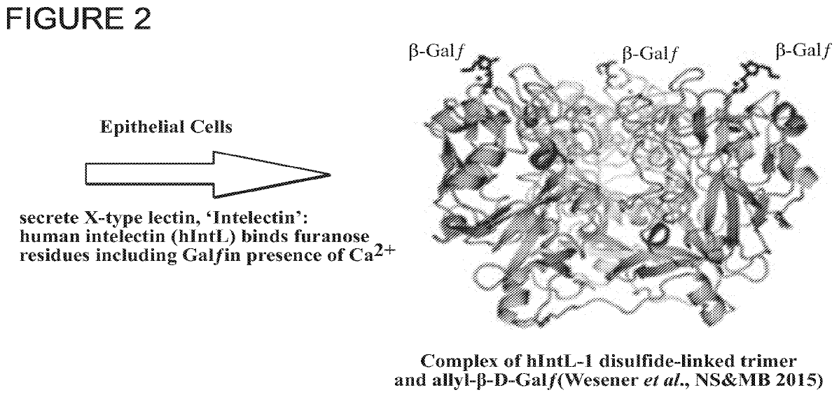

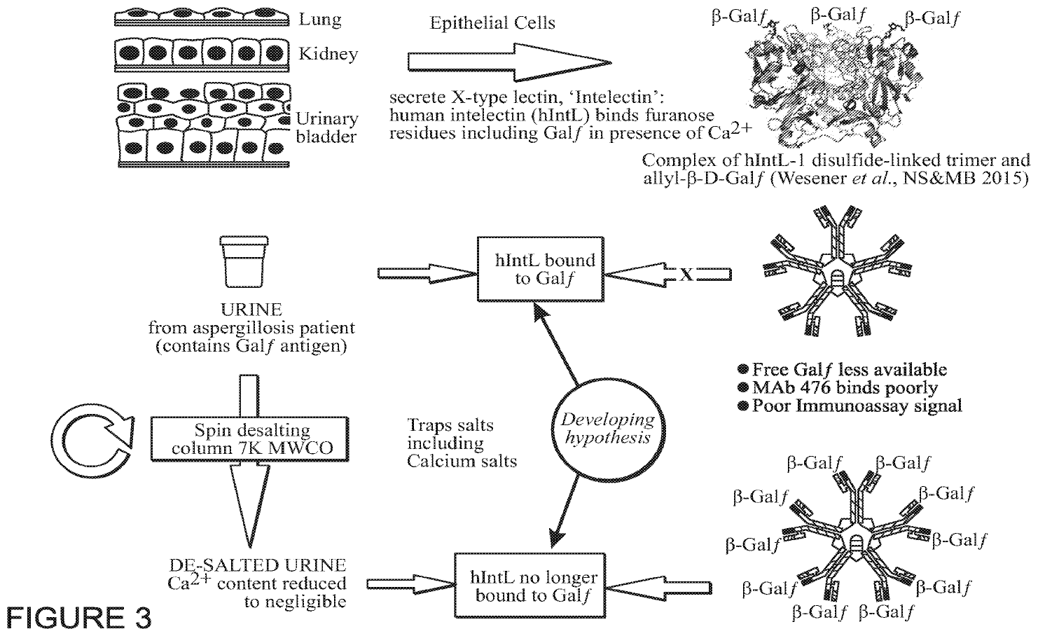

### DETAILED DESCRIPTION

Embodiments of the invention are directed, inter alia, to methods for improving detection and optimizing the sensitivity of diagnostic assays by inhibiting intelectin interference. Embodiments of the invention are directed, inter alia, to methods for improving detection and optimizing the sensitivity of the antibodies, namely improved detection of galF in urine and other compositions which can bind to galF. Compositions include agents which decrease interference by intelectin and/or reduce or eliminate human intelectin from a sample. Methods, embodied herein, improve sensitivity and performance of such detection assays with minimal sample processing.

The antibody of the presently disclosed methods is, in some aspects, specific for at least one polysaccharide comprising a galactofuranose residue and is selected from the group consisting of monoclonal antibody 205 (MAb 205) comprising a variable heavy (VH) domain of SEQ ID NO:1 and a variable light (VL) domain of SEQ ID NO:2; monoclonal antibody 24 (MAb 24) comprising a VH domain of SEQ ID NO:3 and a VL domain of SEQ ID NO:4; monoclonal antibody 686 (MAb 686) comprising a VH domain of SEQ ID NO:5 and a VL domain of SEQ ID NO:6; monoclonal antibody 838 (MAb 838) comprising a VH domain of SEQ ID NO:7 and a VL domain of SEQ ID NO:8; and monoclonal antibody 476 (MAb 476) comprising a VH domain of SEQ ID NO:9 and a VL domain of SEQ ID NO:10. The presently disclosed methods are suitable for use in biological samples including but not limited to urine, bronchoalveolar lavage (BAL) fluid, saliva, serum, gastrointestinal fluids, blood, and cerebrospinal fluid (CSF).

In other embodiments, the presently disclosed subject matter provides a lateral flow device adapted to perform the presently disclosed methods for diagnosing a microbial infection in a biological sample of a mammalian subject suspected of having, having, or susceptible to having a microbial infection.

In accordance with some embodiments, presently disclosed subject matter provides a dipstick assay to perform the presently disclosed methods for diagnosing a microbial infection in a biological sample of a mammalian subject suspected of having, having, or susceptible to having a microbial infection.

In yet other embodiments, the presently disclosed subject matter provides an antibody specific for at least epitope of a polysaccharide secreted by a microbial organism, wherein in particular aspects, the polysaccharide comprises a galactofuranose residue.

Point-of-Care Diagnostics Such as Lateral Flow Devices and Dipstick Assays: Theory and Current Applications.

The ability to provide test results rapidly to the patient and/or healthcare provider is very important to impact outcomes of multiple conditions. Rapid tests to aid diagnosis and enable early detection of multiple diseases and physiologic conditions are being developed. Such tests are especially useful when they can be applied with self-testing and require little in the way of laboratory processing. Examples of point-of-care (POC) test devices in common use today include pregnancy and fertility tests, as well as assays to follow blood glucose in diabetics. Development of diagnostic tests for infections that use POC testing are especially important in resource-poor settings; for this reason, POC testing has become a new goal to be achieved for infections such as HIV, malaria, and hepatitis. Similarly, POC testing has the potential of impacting clinical outcomes when applied to infections that occur in the outpatient setting, not only by providing indications of disease, but by enabling development of more robust prevention algorithms.

Commonly used immunoassays in diagnostic and research use include radio-immunoassays and enzyme-linked immunosorbent assays (ELISAs). Many of these elaborately configured immunoassays use monoclonal antibodies (mAbs) that possess the ability to bind specifically to the analyte being tested, thereby enhancing the accuracy of the assay. Various approaches have been described for carrying out enzyme immunoassays. A considerable number of these approaches, starting with the earliest of ELISAs, are solid-phase immunoassays in which the analyte to be detected is bound to a solid matrix directly (Direct ELISA) or indirectly (Sandwich ELISA), in which the analyte is captured on a primary reagent. The choice of the solid matrix depends on procedural considerations. A common matrix is the polystyrene surface of multi-well microtiter plates.

These types of assays also are amenable to developing POC devices, in which systems can be self-contained so that output is readable by the user. This characteristic is especially useful when collection of a sample to be tested does not require medical intervention (e.g., urine, saliva, or sputum). One device that enables this is the lateral-flow device (LFD). These devices use a multi-layered construction containing both absorbent and non-absorbent components to form a solid-phase. The capture and/or recognition reagents (antigen or antibody) are pre-applied to specific areas within the assembled apparatus and the analyte is allowed to flow through the system to come into contact with reagents. Often, for the purpose of self-containment, the reagent components are added in a dried state so that fluid from the sample re-hydrates and activates them. Conventional ELISA techniques can then be used to detect the analyte in the antigen-antibody complex. In some embodiments, the system can be designed to provide a colorimetric reading for visual estimation of a binary response (‘yes’ or ‘no’), or it can be configured to be quantitative.

Lateral flow devices are used to detect analytes in multiple body fluids, including serum and urine. To date, these types of devices have seen the most use for detecting circulating endogenous analytes; perhaps the most common use of this type of device is in the ubiquitous POC pregnancy test. Current efforts are being directed toward detecting microbial analytes, including nucleic acids, in the setting of viral infections (e.g., influenza, respiratory syncytial virus, and the like), Nielsen, K., et al., Prototype single step lateral flow technology for detection of avian influenza virus and chicken antibody to avian influenza virus. J Immunoassay Immunochem, 2007. 28(4): p. 307-18; Mokkapati, V. K., et al., Evaluation of UPlink-RSV: prototype rapid antigen test for detection of respiratory syncytial virus infection. Ann N Y Acad Sci, 2007. 1098: p. 476-85; bacterial infections (e.g., S. pneumoniae, Legionella, Mycobacteria), Koide, M., et al., Comparative evaluation of Duopath Legionella lateral flow assay against the conventional culture method using Legionella pneumophila and Legionella anisa strains. Jpn J Infect Dis, 2007. 60(4): p. 214-6.

One assay that is in use worldwide is the BinaxNOW pneumococcal urinary antigen test; this assay evolved after the serum-based platform was shown to be effective, but cumbersome. The urinary POC device can be particularly useful when employed in high-risk patients as a POC testing device. Roson, B., et al., Contribution of a urinary antigen assay (Binax NOW) to the early diagnosis of pneumococcal pneumonia. Clin Infect Dis, 2004. 38(2): p. 222-6; Weatherall, C., R. Paoloni, and T. Gottlieb, Point-of-care urinary pneumococcal antigen test in the emergency department for community acquired pneumonia. Emerg Med J, 2008. 25(3): p. 144-8. This issue is particularly relevant in the context of the presently disclosed subject matter, as the polysaccharides in the pneumococcus capsule have some structural similarity to those of Aspergillus. Kappe, R. and A. Schulze-Berge, New cause for false-positive results with the Pastorex Aspergillus antigen latex agglutination test. J Clin Microbiol, 1993. 31(9): p. 2489-90; Stynen, D., et al., Rat monoclonal antibodies against Aspergillus galactomannan. Infect Immun, 1992. 60(6): p. 2237-45; Swanink, C. M., et al., Specificity of a sandwich enzyme-linked immunosorbent assay for detecting Aspergillus galactomannan. J Clin Microbiol, 1997. 35(1): p. 257-60.

Lateral Flow Device and Optimized Methods of Use Thereof for Diagnosing Microbial Infections.

Preliminary studies have demonstrated that polysaccharide antigens of A. fumigatus (e.g., galF) are renally concentrated in animal model and are excreted in urine such that the sensitivity and specificity of a urine-based assay may equal or exceed that of serum based testing. Urinary detection of antigens would enable development of an easy-to-use POC testing method that would enable frequent testing in the outpatient setting, thus aiding the ability to diagnose and optimize screening strategies employed to detect infection early in the course of disease. Accordingly, in some embodiments, the presently disclosed subject matter provides a POC test to detect Aspergillus galF-containing antigens in urine. Monoclonal antibodies that recognize galactofuranose residues of A. fumigatus galF have been developed and are used in the presently disclosed galF test.

A standard ELISA format was used as a screen to identify antibodies to use for capture on the immobilized device. The identified antibody can be used as a capture antibody with point of care testing device (strip), which can be optimized for conditions to detect galF-antigen (antibody concentration, incubation conditions, and the like).

The term “dipstick assay” as used herein means any assay using a dipstick in which sample solution is contacted with the dipstick to cause sample solution to move by capillary action to a capture zone of the dipstick thereby allowing a target antigen in the sample solution to be captured and detected at the capture zone. To test for the presence of analyte, the contact end of the dipstick is contacted with the test solution. If analyte is present in the test solution it travels to the capture zone of the dipstick by capillary action where it is captured by the capture antibody. The presence of analyte at the capture zone of the dipstick is detected by a further anti-analyte antibody (the detection antibody) labelled with, for example, colloidal gold.

These dipstick tests have several advantages. They are easy and cheap to perform, no specialist instruments are required, and the results are obtained rapidly and can be read visually. These tests are, therefore, particularly suited for use in a physician's office, at home, in remote areas, and in developing countries where specialist equipment may not be available. They can be used, for example, to test whether a patient is infected with a disease causing micro-organism such as A. fumigatus.

To perform a method of the first aspect of the invention, the targeting agent and labels may simply be added to the test solution and the test solution then contacted with the contact end of the chromatographic strip. Such methods are easier to perform than the method disclosed in WO 00/25135 in which two separate wicking steps are required. The results may, therefore, be obtained more rapidly, and yet the sensitivity of analyte detection is higher.

The term “chromatographic strip” is used herein to mean any porous strip of material capable of transporting a solution by capillarity. The chromatographic strip may be capable of bibulous or non-bibulous lateral flow, but preferably bibulous lateral flow. By the term “non-bibulous lateral flow” is meant liquid flow in which all of the dissolved or dispersed components of the liquid are carried at substantially equal rates and with relatively unimpaired flow laterally through the membrane as opposed to preferential retention of one or more components as would occur with “bibulous lateral flow.” Materials capable of bibulous lateral flow include paper, nitrocellulose, and nylon. A preferred example is nitrocellulose.

The labels may be bound to the ligands of the targeting agent by pre-mixing the targeting agent with the labels before the targeting agent is added to (or otherwise contacted with) the test solution. However, in some circumstances, it is preferred that the targeting agent and labels are not pre-mixed because such pre-mixing can cause the targeting agent and labels to precipitate. Thus, the targeting agent and the labels may be added separately to (or contacted separately with) the test solution. The targeting agent and the labels can be added to (or contacted with) the test solution at substantially the same time, or in any order.

The test solution may be pre-incubated with the targeting agent and labels before the test solution is contacted with the contact end of the chromatographic strip to ensure complex formation. The optimal time of pre-incubation will depend on the ratio of the reagents and the flow rate of the chromatographic strip. In some cases, pre-incubation for too long can decrease the detection signal obtained, and even lead to false positive detection signals. Thus, it may be necessary to optimize the pre-incubation time for the particular conditions used.

It may be desired to pre-incubate the targeting agent with the test solution before binding the labels to the targeting agent so that the targeting agent can be allowed to bind to analyte in the test solution under optimum binding conditions.

Generally, the presently disclosed subject matter provides a method for diagnosing a microbial infection in a biological sample from a mammalian subject suspected of having, having, or susceptible to having a microbial infection by detecting the presence of at least one polysaccharide comprising a galF residue in a biological sample of the mammalian subject, the method comprising: (a) treating the biological sample to inhibit human intelectin-1 (hIntL-1) binding of galF residues present in the sample; (b) contacting the treated sample of (a) with at least one antibody specific for at least one polysaccharide comprising a galF residue in an effective amount to produce a detectable amount of antibody-polysaccharide complex; and (c) detecting the presence of at least one antibody-polysaccharide complex, wherein the detection of the presence of at least one antibody-polysaccharide complex is diagnostic of a microbial infection in a mammalian subject.

In accordance with another embodiment, the present invention provides a method for diagnosing a microbial infection in a biological sample from a mammalian subject suspected of having, having, or susceptible to having a microbial infection by detecting the presence of at least one polysaccharide comprising a galF residue in a biological sample of the mammalian subject, the method comprising: (a) treating the biological sample comprising contacting the sample with a substrate which binds mono and divalent cations with high affinity, to inhibit human intelectin (hIntL) binding of galF residues present in the sample; (b) contacting the treated sample of (a) with at least one antibody specific for at least one polysaccharide comprising a galF residue in an effective amount to produce a detectable amount of antibody-polysaccharide complex; and (c) detecting the presence of at least one antibody-polysaccharide complex, wherein the detection of the presence of at least one antibody-polysaccharide complex is diagnostic of a microbial infection in a mammalian subject.

Though not wishing to be bound by the following theory, it is thought that hIntL-1 exhibits high affinity and selectivity for β-linked D-galactofuranose residues (Gale, D-phosphoglycerol-modified glycans, heptoses, D-glycero-D-talo-oct-2-ulosonic acid (KO) and 3-deoxy-D-manno-oct-2-ulosonic acid (KDO) containing glycans. The 1.6 Å resolution crystal structure of hIntL-1 bound to β-Galf revealed that hIntL-1 uses a bound calcium ion to coordinate terminal exocyclic 1,2-diols. N-Acetylneuraminic acid (Neu5Ac), a sialic acid widespread in human glycans, possesses an exocyclic 1,2-diol but does not bind hInt-1, likely due to unfavorable steric and electronic effects. Human IntL-1 marks Streptococcus pneumoniae serotypes that display surface glycans with terminal 1,2-diol groups (D. A. Wesener et al., “Recognition of Microbial Glycans by Human Intelectin”, Nat Struct Mol Biol. 2015 August; 22(8): 603-610).

In accordance with certain embodiments, a method of detecting a microorganism in a sample, comprises: (a) contacting or treating the sample with one or more compounds comprising: glycerol, 3-Keto-2-deoxyoctonic acid (KDO), D-glycerol-1-phosphate, D-mannoheptose lactoferrin, saccharides, D-phospho-glycerol-modified glycans, heptoses, D-glycero-D-talo-oct-2-ulosonic acid (KO), 3-deoxy-D-manno-oct-2-ulosonic acid (KDO), sepharose, variants, derivatives or combinations thereof; (b) contacting the treated sample with an antibody which specifically binds to polysaccharides comprising galactofuranose; (c) detecting the presence of at least one antibody-polysaccharide complex, thereby detecting the microorganism in the sample. In a particular aspect, the inventors use sepharose or variants thereof to remove intelectin from the biological sample, e.g. urine. In certain embodiments, the one or more compounds are in solution or immobilized on a support or packed in a column.

Another method that can be employed to remove, eliminate or decrease the amount of intelectin to interfere in an assay is using affinity to carbohydrates. Specifically for hIntL-1, linear carbohydrates (specifically sorbitol), or other carbohydrates that contain an exocyclic diol (exclusive of β-galactofuranose), can be immobilized on a resin. The terminal exocyclic diol on most linear carbohydrates is an excellent ligand for intelectins, so when they are immobilized on a resin, they capture intelectins in a calcium ion dependent manner. If required, they can be eluted by EDTA or the addition of excess exocyclic diol containing compounds (such as glycerol or sorbitol).

In certain embodiments, a method of removing, eliminating or decreasing the amount of human intelectin molecules from a biological sample, comprises contacting the biological sample containing the human intelectin with a one or more compounds comprising a linear carbohydrate or carbohydrate comprising an exocyclic diol under conditions permitting binding of the human intelectin to the linear carbohydrate. In certain embodiments, the exocyclic diol is exclusive of galactofuranose. In certain embodiments, the linear carbohydrate or carbohydrate comprising an exocyclic diol is in solution or immobilized on a support or packed in a column. In certain embodiments, the support is a dipstick, bead, chip, microwell, filter, resin, membrane, or quantum dot.

The microbial infection can be selected from the group consisting of a bacterial infection and a fungal infection. In some embodiments, the microbial infection is caused by an organism comprising: Streptococcus pneumoniae, Klebsiella pneumonia, Escherichia coli, Mycobacteria species, Malassezia species, Aspergillus species, Fusarium species, Alternaria species, Coccidioides species, Cryptococcus species, Mucormycetes, Histoplasma species, Neosartorya species, Fusarium species, Paracoccidioides species, or combinations thereof.

In particular embodiments, at least one antibody specific for at least one polysaccharide comprising a galactofuranose residue comprises: monoclonal antibody 205 (MAb 205) comprising a variable heavy (VH) domain of SEQ ID NO:1 and a variable light (VL) domain of SEQ ID NO:2; monoclonal antibody 24 (MAb 24) comprising a VH domain of SEQ ID NO:3 and a VL domain of SEQ ID NO:4; monoclonal antibody 686 (MAb 686) comprising a VH domain of SEQ ID NO:5 and a VL domain of SEQ ID NO:6; monoclonal antibody 838 (MAb 838) comprising a VH domain of SEQ ID NO:7 and a VL domain of SEQ ID NO:8; and monoclonal antibody 476 (MAb 476) comprising a VH domain of SEQ ID NO:9 and a VL domain of SEQ ID NO:10, or combinations thereof.

One of ordinary skill in the art upon review of the presently disclosed subject matter would appreciate that any biological fluid in which at least one polysaccharide comprising a galactofuranose residue is secreted is suitable for use with the presently disclosed methods. In particular embodiments, the biological sample comprises urine, bronchoalveolar lavage (BAL) fluid, saliva, serum, gastrointestinal fluids, blood, or cerebrospinal fluid (CSF).

In some embodiments, the presently disclosed methods further comprise pre-treating the biological sample before contacting the biological sample with at least one antibody specific for at least one polysaccharide comprising a galactofuranose residue. The pre-treating step can include a step selected from the group consisting of filtering, diluting, and concentrating the biological sample, and combinations thereof.

In accordance with some embodiments, the method for treating the sample in step (a) comprises contacting the sample with a substrate which binds Ca′ ions with high affinity.

Examples of substrates which can bind divalent cations with high affinity include, for example, N,N,N′,N′-tetrakis-(2-pyridylmethyl)ethylenediamine (TPEN, membrane-permeable chelator) and diethylenetriaminepentaacetic acid (DTPA, membrane-impermeable chelator), cation exchange resins such as AG50, Chelex, poly(acrylic acid), and others, such as sepharose, for example.

In accordance with some embodiments, the method for treating the sample in step (a) comprises contacting the sample with a compound which chelates Ca²⁺ ions with high affinity. Examples of chelators include, without limitation, ethylenediamine tetraacetic acid (EDTA), Ethylene glycol-bis(2-aminoethylether)-N,N,N′,N′-tetraacetic acid (EGTA), 1,2-bis(o-Aminophenoxy)ethane-N,N,N′,N′-tetraacetic Acid (BAPTA), 1-(2-Nitro-4,5-dimethoxyphenyl)-1,2-diaminoethane-N,N,N′,N′-tetraacetic Acid, 4Na, Dimethoxynitrophenamine (DM-Nitrophen), and others.

In accordance with some embodiments, the method for treating the sample in step (a) comprises contacting the sample with EDTA and/or EGTA.

In accordance with some embodiments, the method for treating the sample in step (a) comprises contacting the sample with a substrate which binds hIntL-1 with high affinity.

Examples of compounds which bind hIntL-1 include, but are not limited to, glycerol, 3-keto-2-deoxyoctonic acid, D-glycerol-1-phosphate, D-mannoheptose, lactoferrins, saccharides, D-phospho-glycerol-modified glycans, heptoses, D-glycero-D-talo-oct-2-ulosonic acid (KO), 3-deoxy-D-manno-oct-2-ulosonic acid (KDO), sepharose and other compounds which are bound by hIntL-1.

In accordance with some embodiments, the method for treating the sample in step (a) comprises contacting the sample with an antibody specific for hIntL-1. In some embodiments, the antibody can be rabbit polyclonal IgG anti-human INTL-1 antibody.

In accordance with some embodiments, the method for treating the sample in step (a) comprises contacting the sample with one or more compounds which are bound by hIntL-1 with high affinity.

In accordance with some embodiments, the method for treating the sample in step (a) comprises contacting the sample with one or more compounds which bind hIntL with high affinity comprise glycerol, 3-Keto-2-deoxyoctonic acid; D-glycerol-1-phosphate, D-mannoheptose lactoferrins, lectins, saccharides, D-phospho-glycerol-modified glycans, heptoses, D-glycero-D-talo-oct-2-ulosonic acid (KO), 3-deoxy-D-manno-oct-2-ulosonic acid (KDO), sepharose or combinations thereof.

In accordance with some embodiments, the method for treating the sample in step (a) comprises a combination of one or more of the above methods including, for example, treating the sample with a chelator and one or more compounds which are bound by hIntL-1 with high affinity, and an anti-IntL-antibody. Any of the above methods can be combined to further prevent hIntL-1 from binding galF in a biological sample.

In accordance with some embodiments, the method for treating the sample in step (a) comprises contacting the sample with a desalting column. Examples of desalting columns are known in the art, including, for example, desalting columns which are pre-packed with polyacrylamide size exclusion resins.

In accordance with some embodiments, the method for treating the sample in step (a) comprises contacting the sample with a column comprising sepharose or variations thereof. Examples of variations or types of sepharose, include, without limitation beads, resins, gels, coated sepharose beads (e.g. protein A/G coated, heparin coated, etc) and the like are commercially available.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In particular embodiments, the subject is a human adult suspected of having, having, or susceptible of having a microbial infection. In other embodiments, the subject is a human child, e.g., a human less than about 19 years of age, suspected of having, having, or susceptible of having a microbial infection.

The presently disclosed methods can be used to diagnose, for the prognosis, or the monitoring of a disease state or condition. As used herein, the term “diagnosis” refers to a predictive process in which the presence, absence, severity or course of treatment of a disease, disorder or other medical condition is assessed. For purposes herein, diagnosis also includes predictive processes for determining the outcome resulting from a treatment. Likewise, the term “diagnosing,” refers to the determination of whether a sample specimen exhibits one or more characteristics of a condition or disease. The term “diagnosing” includes establishing the presence or absence of, for example, a target antigen or reagent bound targets, or establishing, or otherwise determining one or more characteristics of a condition or disease, including type, grade, stage, or similar conditions. As used herein, the term “diagnosing” can include distinguishing one form of a disease from another. The term “diagnosing” encompasses the initial diagnosis or detection, prognosis, and monitoring of a condition or disease.

The term “prognosis,” and derivations thereof, refers to the determination or prediction of the course of a disease or condition. The course of a disease or condition can be determined, for example, based on life expectancy or quality of life. “Prognosis” includes the determination of the time course of a disease or condition, with or without a treatment or treatments. In the instance where treatment(s) are contemplated, the prognosis includes determining the efficacy of a treatment for a disease or condition.

As used herein, the term “risk” refers to a predictive process in which the probability of a particular outcome is assessed. The term “monitoring,” such as in “monitoring the course of a disease or condition,” refers to the ongoing diagnosis of samples obtained from a subject having or suspected of having a disease or condition. The term “marker” refers to a molecule, including an antigen, such as a polysaccharide, that when detected in a sample is characteristic of or indicates the presence of a disease or condition.

Accordingly, in some embodiments, the presently disclosed subject matter provides a method for diagnosing of a microbial infection in a mammalian subject suspected of having, having, or susceptible to having a microbial infection, wherein the method comprises monitoring a treatment regimen of a microbial infection to determine the efficacy of the treatment regimen.

In accordance with some embodiments, the methods disclosed herein can be used with lateral flow devices such as those disclosed in U.S. patent application Ser. No. 13/511,264, and incorporated by reference herein in its entirety. The presently disclosed methods can use a lateral flow device or dipstick assay comprising an immunochromatographic strip test that relies on a direct (double antibody sandwich) reaction. Without wishing to be bound to any one particular theory, this direct reaction scheme is best used when sampling for larger analytes that may have multiple antigenic sites. Different antibody combinations can be used, for example different antibodies can be included on the capture (detection) line, the control line, and included in the mobile phase of the assay, for example, as conjugated to gold particles, e.g., gold microparticles, gold nanoparticles, or fluorescent dyes.

In an embodiment, the present disclosure comprises kits for diagnosing a microbial infection in a biological sample from a mammalian subject suspected of having, having, or susceptible to having a microbial infection by detecting the presence of at least one polysaccharide comprising a galactofuranose residue in a biological sample of the mammalian subject. Such kits may include all necessary reagents, components, apparatus and instructions for treating the biological sample to inhibit human intelectin (hIntL-1) binding of galactofuranose residues present in the sample; in an embodiment, the kits may further comprise at least one antibody specific for at least one polysaccharide comprising a galactofuranose residue in an effective amount to produce a detectable amount of antibody-polysaccharide complex; in an embodiment, the kit further enables detecting the presence of at least one antibody-polysaccharide complex, wherein the detection of the presence of at least one antibody-polysaccharide complex is diagnostic of a microbial infection in a mammalian subject. In certain embodiments, the kit comprises the use of a lateral flow apparatus, dipstick, assay stick with immunochromatographic detection display, and any such apparatus know to those skilled in the art. In certain embodiments, reagents and/or detection components may be immobilized on the apparatus itself (i.e. on the dipstick). In certain embodiments, reagents for chelating calcium are included in the kit.

As used herein the term “lateral flow” refers to liquid flow along the plane of a substrate or carrier, e.g., a lateral flow membrane. In general, lateral flow devices comprise a strip (or a plurality of strips in fluid communication) of material capable of transporting a solution by capillary action, i.e., a wicking or chromatographic action, wherein different areas or zones in the strip(s) contain assay reagents, which are either diffusively or non-diffusively bound to the substrate, that produce a detectable signal as the solution is transported to or migrates through such zones. Typically, such assays comprise an application zone adapted to receive a liquid sample, a reagent zone spaced laterally from and in fluid communication with the application zone, and a detection zone spaced laterally from and in fluid communication with the reagent zone. The reagent zone can comprise a compound that is mobile in the liquid and capable of interacting with an analyte in the sample, e.g., to form an analyte-reagent complex, and/or with a molecule bound in the detection zone. The detection zone may comprise a binding molecule that is immobilized on the strip and is capable of interacting with the analyte and/or the reagent and/or an analyte-reagent complex to produce a detectable signal. Such assays can be used to detect an analyte in a sample through direct (sandwich assay) or competitive binding. Examples of lateral flow devices are provided in U.S. Pat. No. 6,194,220 to Malick et al.; U.S. Pat. No. 5,998,221 to Malick et al.; U.S. Pat. No. 5,798,273 to Shuler et al.; and RE38,430 to Rosenstein.

In some embodiments, the presently disclosed methods can be used with an assay comprising a sandwich lateral flow or dipstick assay. In a sandwich assay, a liquid sample that may or may not contain an analyte of interest is applied to the application zone and allowed to pass into the reagent zone by capillary action. The term “analyte” as used herein refers to a polysaccharide comprising a galactofuranose residue. In certain embodiments the presence or absence of an analyte in a sample is determined qualitatively. In other embodiments, a quantitative determination of the amount or concentration of analyte in the sample is determined.

The analyte, if present, interacts with a labeled reagent in the reagent zone to form an analyte-reagent complex and the analyte-reagent complex moves by capillary action to the detection zone. The analyte-reagent complex becomes trapped in the detection zone by interacting with a binding molecule specific for the analyte and/or reagent. Unbound sample can pass through the detection zone by capillary action to a control zone or an absorbent pad laterally juxtaposed and in fluid communication with the detection zone. The labeled reagent may then be detected in the detection zone by appropriate means.

Generally, and without limitation, lateral flow devices comprise a sample pad. A sample pad comprises a membrane surface, also referred to herein as a “sample application zone,” adapted to receive a liquid sample. A standard cellulose sample pad has been shown to facilitate absorption and flow of biological samples, including, but not limited to, urine. The sample pad comprises a portion of lateral flow device that is in direct contact with the liquid sample, that is, it receives the sample to be tested for the analyte of interest. The sample pad can be part of, or separate from, a lateral flow membrane. Accordingly, the liquid sample can migrate, through lateral or capillary flow, from sample pad toward a portion of the lateral flow membrane comprising a detection zone. The sample pad is in fluid communication with the lateral flow membrane comprising an analyte detection zone. This fluid communication can arise through either be an overlap, top-to-bottom, or an end-to-end fluid connection between the sample pad and a lateral flow membrane. In certain embodiments, the sample pad comprises a porous material, for example and not limited to, paper.

The term “sample” as used herein refers to any biological sample suspected of containing an analyte for detection or a control sample expected to be substantially free of the analyte of interest. In particular embodiments, the sample comprises a biological fluid of a subject suspected of having, having, or susceptible of having a microbial infection. In some embodiments, the biological sample is in liquid form, while in other embodiments it can be changed into a liquid form, e.g., by reconstitution in a suitable solvent, e.g., an aqueous solution. The presently disclosed lateral flow devices are suitable for use with a variety of biological samples including, but not limited to, urine, bronchoalveolar lavage (BAL) fluid, serum, blood, gastrointestinal fluids, and cerebrospinal fluid (CSF).

Typically, a sample pad is positioned adjacent to and in fluid communication with a conjugate pad. A conjugate pad comprises a labeled reagent having specificity for one or more analytes of interest. In some embodiments, the conjugate pad comprises a non-absorbent, synthetic material (e.g., polyester) to ensure release of its contents. A detection conjugate is dried into place on the conjugate pad and only released when the liquid sample is applied to the sample pad. Detection conjugate can be added to the pad by immersion or spraying.

In particular embodiments, the detection conjugate comprises an antibody having specificity for a polysaccharide comprising a galactofuranose residue. In representative embodiments, the antibody comprises: monoclonal antibody 205 (MAb 205) comprising a variable heavy (VH) domain of SEQ ID NO:1 and a variable light (VL) domain of SEQ ID NO:2; monoclonal antibody 24 (MAb 24) comprising a VH domain of SEQ ID NO:3 and a VL domain of SEQ ID NO:4; monoclonal antibody 686 (MAb 686) comprising a VH domain of SEQ ID NO:5 and a VL domain of SEQ ID NO:6; monoclonal antibody 838 (MAb 838) comprising a VH domain of SEQ ID NO:7 and a VL domain of SEQ ID NO:8; and monoclonal antibody 476 (MAb 476) comprising a VH domain of SEQ ID NO:9 and a VL domain of SEQ ID NO:10, or combinations thereof. The antibody, e.g., a monoclonal antibody (MAb), can be conjugated to a fluorescent dye or gold particle, e.g., colloidal gold, including igold microspheres or gold nanoparticles, such as gold nanoparticles of about 40 nm. For example, it is possible to biotinylate the conjugated MAb to take advantage of the strong affinity that biotin has for streptavidin, using Streptavidin-coated microspheres. Alternatives include protein A-coated microspheres that bind to Fc region of IgGs. Conditions to define optimal optimization to colloidal gold can be determined, for example, in microtiter wells. For example, 100 μL of colloidal gold at 1 OD530 can be added to each well, followed by 10 μL of 22 mM buffers (MES, HEPES) at variable pH (5.5 to 10, in 0.5 increments). Antibodies can be added at concentrations ranging from about 1.25 μg/l OD colloid to about 10 μg/l OD colloid, incubated for 15 minutes, and then 25 μL of 1.5 NaCl can be added. Conjugated particles will be stable and pink; the optimal condition that requires the lowest concentration of antibodies can be determined.

Usually, the conjugate pad is adjacent to and in fluid communication with a lateral flow membrane. Capillary action draws a fluid mixture up the sample pad, through the conjugate pad where an antibody-polysaccharide complex is formed, and into the lateral flow membrane. Lateral flow is a function of the properties of the lateral flow membrane. The lateral flow membrane typically is extremely thin and is hydrophilic enough to be wetted, thereby permitting unimpeded lateral flow and mixture of reactants and analytes at essentially the same rates.

Lateral flow membranes can comprise any substrate capable of providing liquid flow including, but not limited to, substrates, such as nitrocellulose, nitrocellulose blends with polyester or cellulose, untreated paper, porous paper, rayon, glass fiber, acrylonitrile copolymer, plastic, glass, or nylon. Lateral flow membranes can be porous. Typically, the pores of a lateral flow membrane are of sufficient size such that particles, e.g., microparticles comprising a reagent capable of forming a complex with an analyte, flow through the entirety of the membrane. Lateral flow membranes, in general, can have a pore size ranging from about 3 μm to about 100 μm, and, in some embodiments, have a pore size ranging from about 10 μm to about 50 μm. Pore size affects capillary flow rate and the overall performance of the device.

There are multiple benefits to using nitrocellulose for the primary membrane: low cost, capillary flow, high affinity for protein biding, and ease of handling. Nitrocellulose has high protein binding. Another alternative is cellulose acetate, which has low protein binding. Size dictating surface area dictates membrane capacity (the volume of sample that can pass through the membrane per unit time=length×width×thickness×porosity. Because these variables control the rate at which lateral flow occurs, they can impact sensitivity and specificity of the assay. The flow rate also varies with sample viscosity. Several different sizes and polymers are available for use as microspheres, which migrate down the membrane with introduction of the fluidic sample. The optimal flow rate generally is achieved using spheres that are 1/10 the pore size of the membrane or smaller.

One skilled in the art will be aware of other materials that allow liquid flow. Lateral flow membranes, in some embodiments, can comprise one or more substrates in fluid communication. For example, a conjugate pad can be present on the same substrate or may be present on separate substrates (i.e., pads) within or in fluid communication with lateral flow membranes. In some embodiments, the nitrocellulose membrane can comprise a very thin Mylar sheet coated with a nitrocellulose layer.

Lateral flow membranes can further comprise at least one indicator zone or detection zone. The terms “indicator zone” and “detection zone” are used interchangeably herein and mean the portion of the carrier or porous membrane comprising an immobilized binding reagent. As used herein, the term “binding reagent” means any molecule or a molecule bound to a particle, wherein the molecule recognizes or binds the analyte in question. The binding reagent is capable of forming a binding complex with the analyte-labeled reagent complex. The binding reagent is immobilized in the detection zone and is not affected by the lateral flow of the liquid sample due to the immobilization on the membrane. Once the binding reagent binds the analyte-labeled reagent complex it prevents the analyte-labeled reagent complex from continuing with the flow of the liquid sample. In some embodiments, the binding reagent is an antibody having specificity for a polysaccharide having at least one galactofuranose residue.

Accordingly, during the actual reaction between the analyte and the reagent, the first member binds in the indicator zone to the second member and the resulting bound complex is detected with specific antibodies. Detection may use any of a variety of labels and/or markers, e.g., enzymes (alkaline phosphatase or horseradish peroxidase with appropriate substrates), radioisotopes, liposomes or latex beads impregnated with fluorescent tags, polymer dyes or colored particles, and the like. Thus, the result can be interpreted by any direct or indirect reaction. Colloidal gold particles, which impart a purple or red coloration, are most commonly used currently.

The capture and immobilization of the assay reagent (complementary member of the binding pair) at the indicator zone can be accomplished by covalent bonding or, more commonly, by adsorption, such as by drying. Such capture also can be indirect, for example, by binding of latex beads coated with the reagent. Depending on the nature of the material comprising the lateral flow membrane, covalent bonding may be enabled, for example with use of glutaraldehyde or a carbodiimide. In immunoassays, most common binding pairs are antigen-antibody pairs; however, multiple other binding pairs can be performed, such as enzyme-substrate and receptor-ligand.

In some embodiments, the indicator zone further comprises a test line and a control line. A test line can comprise an immobilized binding reagent. When antibodies are used to develop a test line in the LFD that employs a sandwich type of assay, they are applied at a ratio of about 1-3 μg/cm across the width of a strip 1 mm wide; hence, antibody concentration is about 10-30 μg/cm², which is about 25-100 fold that used in an ELISA. Brown, M. C., Antibodies: key to a robust lateral flow immunoassay, in Lateral Flow Immunoassay, H. Y. T. R. C. Wong, Editor. 2009, Humana Press: New York, N.Y. p. 59-74.

Further, in some embodiments, the presently disclosed lateral flow assays can be used to detect multiple analytes in a sample. For example, in a lateral flow assay, the reagent zone can comprise multiple labeled reagents, each capable of binding to a different analyte in a liquid sample or a single labeled reagent capable of binding to multiple analytes. If multiple labeled reagents are used in a lateral flow assay, the reagents may be differentially labeled to distinguish different types of analytes in a liquid sample.

It also is possible to place multiple lines of capture antibodies on the membrane to detect different analytes. Combinations of antibodies that detect different epitopes of glycans may optimize specificity if it is found that one antibody performs at a low quantitative limit of detection, yet exhibits some degree of nonspecific binding (or binding to urine components in control animals). One possibility is that the device may be adapted to detect galF and another fungal component to increase the potential spectrum of pathogens detected and to increase specificity of the reaction. Aspergillus species are thought to secrete galF and other fungal components, while glycans from other ‘contaminants’ should not contain other fungal components.

For quality control, typically a lateral flow membrane can include a control zone comprising a control line. The term “control zone” refers to a portion of the test device comprising a binding molecule configured to capture the labeled reagent. In a lateral flow assay, the control zone may be in liquid flow contact with the detection zone of the carrier, such that the labeled reagent is captured on the control line as the liquid sample is transported out of the detection zone by capillary action. Detection of the labeled reagent on the control line confirms that the assay is functioning for its intended purpose. Placement of a control line can be accomplished using a microprocessor controlled TLC spotter, in which a dispenser pump releases a constant volume of reagent across the membrane.

A typical lateral flow device can also comprises an absorbent pad. The absorbent pad comprises an “absorbent material,” which as used herein, refers to a porous material having an absorbing capacity sufficient to absorb substantially all the liquids of the assay reagents and any wash solutions and, optionally, to initiate capillary action and draw the assay liquids through the test device. Suitable absorbent materials include, for example, nitrocellulose, nitrocellulose blends with polyester or cellulose, untreated paper, porous paper, rayon, glass fiber, acrylonitrile copolymer, plastic, glass, or nylon.

In some embodiments, a lateral flow membrane is bound to one or more substantially fluid-impervious sheets, one on either side, e.g., a bottom sheet and a complimentary top sheet with one or more windows defining an application zone and an indicator zone.

A typical lateral flow device also can include a housing. The term “housing” refers to any suitable enclosure for the presently disclosed lateral flow devices. Exemplary housings will be known to those skilled in the art. The housing can have, for example, a base portion and a lid portion. The lid portion can include a top wall and a substantially vertical side wall. A rim may project upwardly from the top wall and may further define a recess adapted to collect a sample from a subject. Suitable housings include those provided in U.S. Pat. No. 7,052,831 to Fletcher et al and those used in the BD Directigen™ EZ RSV lateral flow assay device.

As with the general method described immediately hereinabove, the microbial infection can be selected from the group consisting of a bacterial infection and a fungal infection. In some embodiments, the bacterial infection is caused by an infection of Streptococcus pneumoniae. In particular embodiments, the microbial infection is a fungal infection caused by an infection of an organism selected from the group consisting of Aspergillus species, Fusarium species, Coccidioides species, Cryptococcus species, and Histoplasma species.

In some embodiments, a polysaccharide having a galactofuranose residue can be measured in whole, unconcentrated, or otherwise unprocessed, biological samples using the presently disclosed methods and devices. In other embodiments, the biological sample can be processed, e.g., concentrated, diluted, filtered, and the like, prior to performing the test. The pre-treatment of the urine sample can include diluting the urine sample in an aqueous solution, concentrating the urine sample, filtering the urine sample, or a combination thereof.

One of ordinary skill in the art upon review of the presently disclosed subject matter would appreciate that the pre-treatment steps can be performed in any particular order, e.g., in some embodiments, the sample can be diluted or concentrated and then filtered, whereas in other embodiments, the sample can be filtered and then diluted or concentrated. In particular embodiments, the presently disclosed methods include filtering the urine sample, for example, through a desalting column, to remove an inhibitor that interferes with the detection of antigen in the urine sample. This step can be performed with or without any further dilution or concentration of the sample.

Thus, in some embodiments, the lateral flow device further comprises an apparatus adapted to pre-treat the biological sample before contacting the biological sample with at least one antibody specific for at least one polysaccharide comprising a galF residue. In particular embodiments, the apparatus is adapted to filter, dilute, or concentrate the biological sample, or combinations thereof. More particularly, the apparatus can be adapted to remove an inhibitor that interferes with the detection of the at least one polysaccharide comprising a galF residue in the biological sample, in particular, a urine sample.

In other embodiments, different parameters of the test, e.g., incubation time, can be manipulated to increase sensitivity and/or specificity of the test to eliminate the need for processing the biological sample.

Accordingly, in some embodiments, the presently disclosed subject matter provides an antibody specific for at least epitope of a polysaccharide secreted by a microbial organism. In particular embodiments, the polysaccharide comprises a galF residue. In more particular embodiments, the antibody is specific for at least one epitope of a polysaccharide secreted by a microbial organism selected from the group consisting of Aspergillus species, Fusarium species, Coccidioides species, Cryptococcus species, Histoplasma species, and certain Streptococcus species.

This invention is further illustrated by the following examples which should not be construed as limiting. By their citation of various references in this document, applicants do not admit any particular reference is “prior art” to their invention. The contents of all references, patents, and published patent applications cited throughout this application, as well as the figures and the sequence listing, are hereby incorporated by reference.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Recombinant IntL-1 Binds to Aspergillus Ethanol Precipitate and Whole Cells (Conidia and Hyphae).

Method for Ethanol Precipitate binding.

Wells of a microtiter plate were coated with 10 μg/ml of EP antigen and blocked with a blocking buffer containing 0.1% BSA in IntL-ELISA buffer (20 mM HEPES pH7.4; 150 mM NaCl; 10 mM CaCl₂); 0.1% Tween-20. Ref: Wesener et al. (2015) Nature Structural & Molecular Biology, 22(8):603). Recombinant human IntL-1 (rintL-1) was serially diluted tenfold in blocking buffer starting at 10⁻⁶ M, and incubated on the EP-coated plate for 2 h at 30° C., followed by washes with IntL-ELISA buffer. The bound IntL was detected by an anti-omentin Rabbit IgG ab (Millipore) diluted 1:1000 in blocking buffer and incubated at 30° C. for 2 h. Wells were again washed and incubated with a goat anti-Rabbit IgG-AP at 1:1000 for 1 h at 30° C. Following another wash, the color was developed by AP substrate for 30 minutes at 37° C.

Method for Conidia & Hyphae Binding.

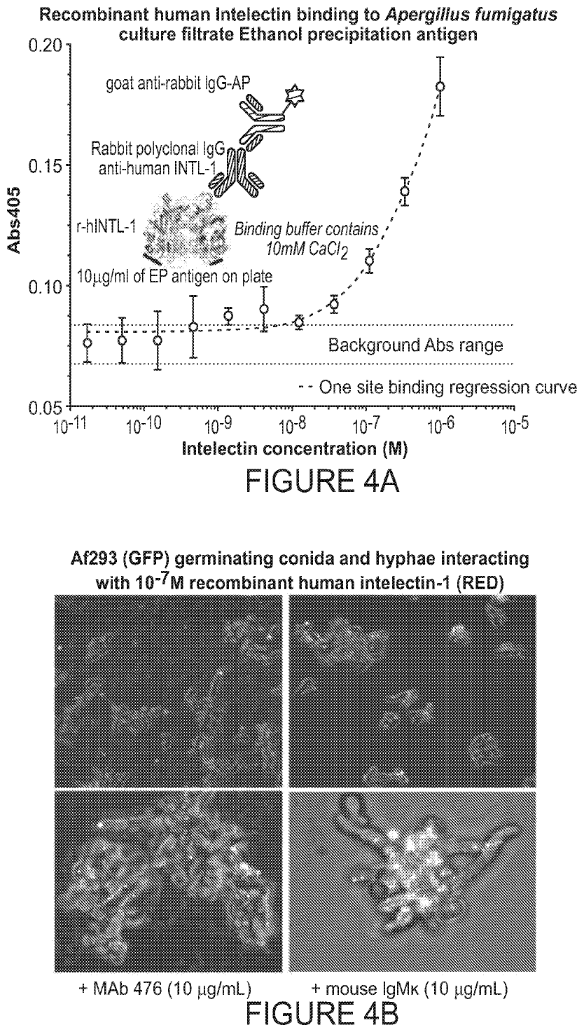

Conidia from Aspergillus fumigatus were counted and adjusted to 10⁶/mL in Sabouraud's Dextrose broth, and grown for 6 hours at 37° C. to germinate (forming hyphae). The collection of swollen, germinating conidia and growing hyphae from already-germinated conidia were killed with heat at 90° C. for 1 h. The fungal material (killed conidia and hyphae) was pelleted by centrifugation, resuspended in IntL-ELISA blocking buffer, and incubated with 10⁻⁷ M rIntL-1 (FLAG-tagged) in presence of 10 μg/mL of either MAb476 or an irrelevant, isotype control antibody mouse IgM for 2 h at 30° C. Following washes with IntL-ELISA buffer, the material was resuspended in IntL-ELISA blocking buffer and incubated with a 1:50 diluted anti-FLAG tag antibody conjugated to the red fluorochrome phycoerythrin (PE). Again, after washes, the stained material was resuspended in IntL-ELISA blocking buffer, transferred to chamber slides and observed under a fluorescent microscope. The inventors performed experiments wherein Aspergillus ethanol precipitate and whole cells (conidia and hyphae) were exposed to rIntL-1 and then a sandwich assay using Rabbit polyclonal antibodies to INTL-1 and goat anti-rabbit antibodies with a fluorescent ligand were performed in buffer comprising 10 mM CaCl₂). As shown in FIG. 4, INTL-1 bound Aspergillus ethanol precipitate with high affinity in the presence of Ca′. Thus, IntL-1 could be the competitive agent which is interfering with the inventors' antibodies to galF.

### Example 2

IntL-1 is Present in Human Urine.

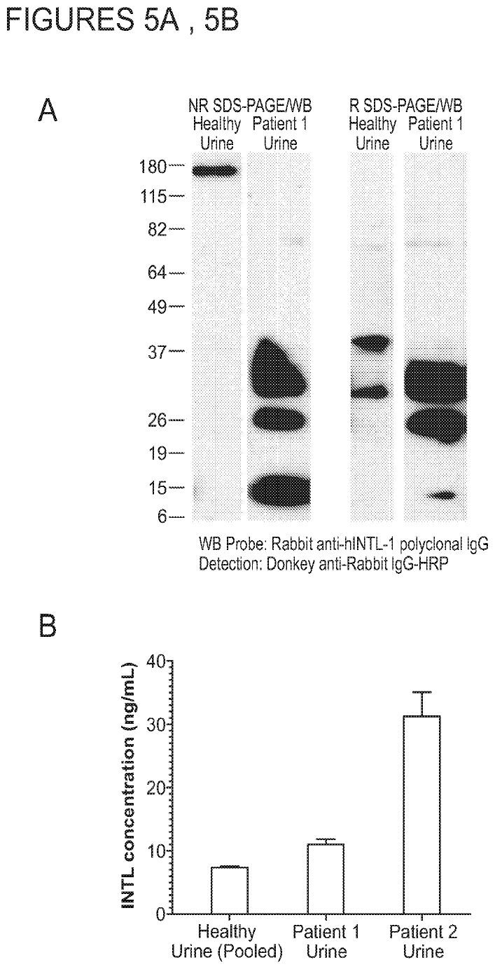

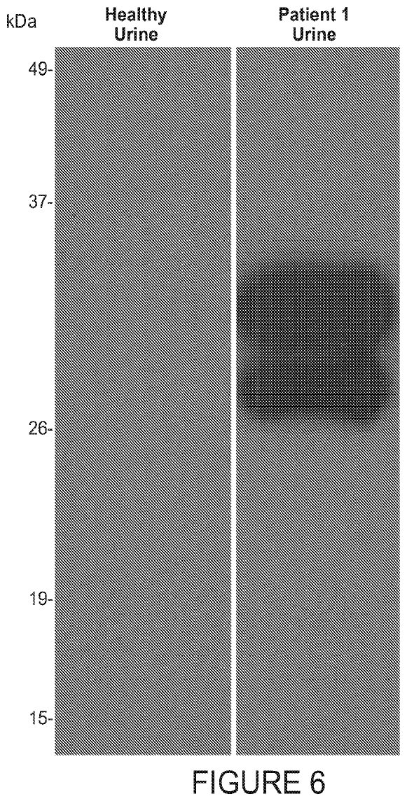

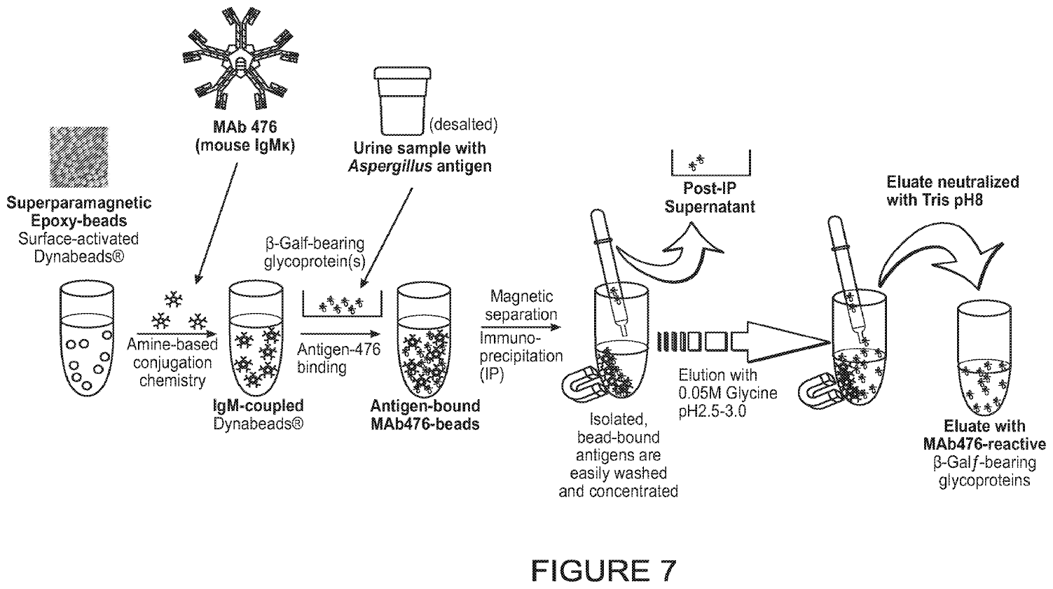

Studies were performed using urine collected from control patients (pool 4) and patient who had Aspergillus infection (VU7). The samples were run in a reducing SDS polyacrylamide gel electrophoresis (PAGE), and the resolved proteins were transferred to a nylon membrane for performing a Western Blot. Some samples used concentrated urine (denoted by “c”) using an Amicon concentrator or were pretreated by desalting columns (denoted by “ds”). As expected in a reducing SDS-PAGE, IntL (trimeric in native state) was resolved to its monomeric form (MW range ˜25-40 kDa). As shown in FIG. 5, IntL-1 was present in patient urine and could also be detected in healthy controls in the concentrated samples. Studies were performed to quantify concentration of hINLT-1 in healthy urine and urine from two patients with aspergillosis, using enzyme immunoassay. Intelectin content of human urine samples was estimated with a double-sandwich ELISA kit (G-Biosciences/Fisher Scientific) for Human ITLN1 following manufacturer's protocol. Provided standards were diluted per protocol and samples were diluted 1:1000 in assay diluent (provided); samples and standards were incubated on antibody coated wells, followed by an HRP-conjugated secondary and an HRP-substrate. The plate wells were read at 450 nm after stopping the reaction, and sample values were extrapolated from a non-linear regression curve based on standard signals.

### Example 3

galF Specific Antibodies Recognize Certain Proteins in Urine.

Studies were performed using urine collected from control patients (pooled) and patient who had Aspergillus infection (VU7). The samples were run in a reducing SDS polyacrylamide gel electrophoresis (PAGE), and the resolved proteins were transferred to a nylon membrane for performing a Western Blot. Some samples used concentrated urine (denoted by “c”) using an Amicon concentrator or were pretreated by desalting columns (denoted by “ds”). Photomicrographs of mAb476 binding showed no reactive bands in the healthy human pooled urine, but small molecular weight bands ranging from 26 to 35 kDa in urine from patients with aspergillosis.

Studies were performed to identify the proteins that are bound by mAb476 after immunoprecipitation, using mass spectrophotometry. To enrich sample detection of moieties specifically binding to mAb476, urines were incubated with superparamagnetic dynabeads coated with mAb476 and a mouse control antibody. mAb476 reactive material was eluted with 50 mM glycine (pH 2.5-3.0). No mAb476 binding occurred in normal urine. Reactivity to mAb476 eluates was confirmed by sandwich ELISA. Eluates were then run on SDS-PAGE gels to check for band patterns, and in-solution mass spectrometry analysis was performed. Analysis detected the microbial antigen CelA/Aspf7 (a galF-containing 0-glycan).

The PEAKS search confirmed the presence of hIntL sequences in the analyzed 476-reactive eluate, indicating the increased likelihood of IntL-1 being the protein in the urine which competitively interferes with mAB476, and binds galF antigens.

### Example 4

Testing of Various Methods to Inhibit Intl-1 Binding to galF Antigens in Patient Urine Samples

The inventors identified IntL-1 as binding to similar molecular weight Aspergillus derived galF-bearing antigens as mAb476 antibody; immunoprecipitation documented that hINTL-1 was bound to Aspergillus O-glycan CelA/Aspf7 along with hINTL-1. Since desalting the urine samples prior to contacting the sample with the galF-reactive antibodies resulted in much greater binding and detection, it was surmised that removal of Ca′ in the samples caused hIntL-1 to lose its native ability to bind galF, making free galF much more available to bind the galF-specific antibodies, such as mAb476. It was therefore thought, that pre-treatment of urine samples with compositions or compounds which either bound free mono and divalent cations, or which served as high-affinity IntL-ligand, would allow the galF specific antibodies bind more of free galF in the samples.

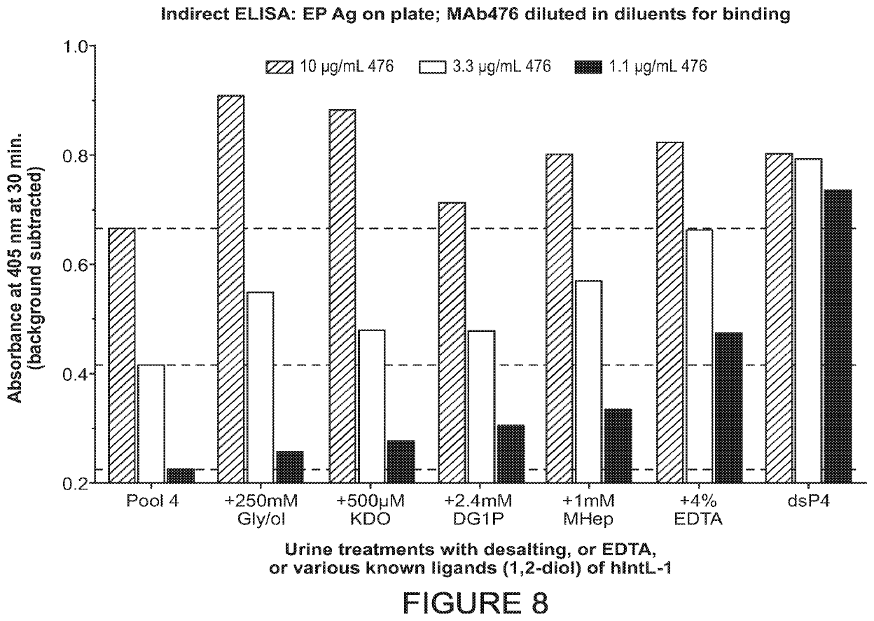

As shown in FIG. 8, in an indirect ELISA assay where Aspergillus ethanol precipitate containing galF is bound to the plate wells, a galF-binding antibody, such as mAB476, was added to the wells diluted in pooled healthy urine (pool 4), untreated or treated with compounds which are known IntL-1 ligands or Ca²⁺ chelators; the samples with the known ligands or chelators had significantly greater antibody binding compared to the untreated urine, and at the highest antibody concentration, the signal was the same or better than the desalting (positive control for treatment). Thus, the inventors have developed a successful methodology to overcome IntL-1 competitive binding without use of extensive pretreatment processing of the urine samples.

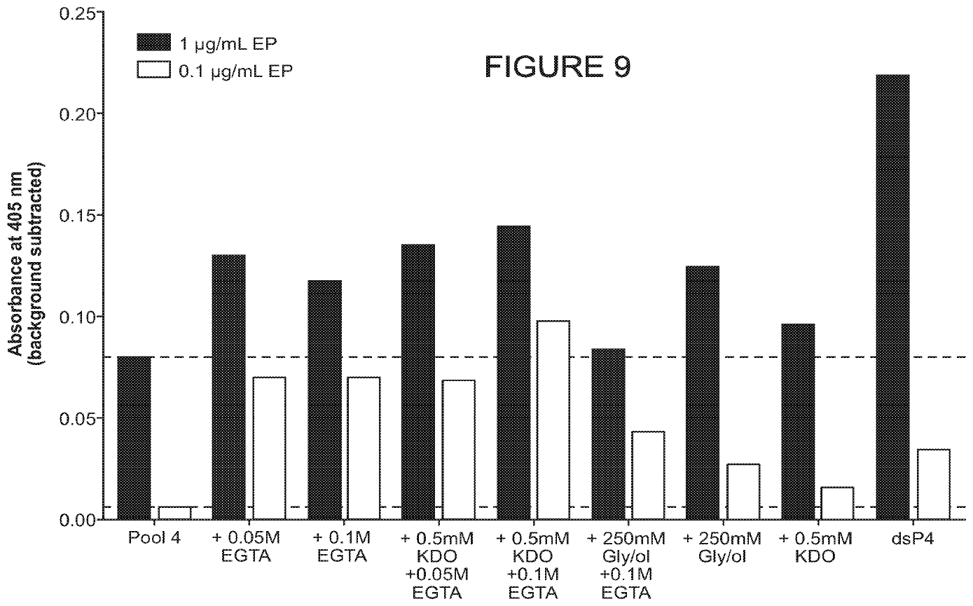

As shown in FIG. 9, in a sandwich ELISA where a galF-binding antibody, such as mAb476, is bound to the plate wells as the capture antibody, low concentrations of Aspergillus ethanol precipitate, namely 0.1 and 1 μg/mL, were diluted in urine, untreated or treated with compounds that are known ligands of IntL-1, or Ca²⁺ chelators, or a combination thereof, incubated with the same galF-binding antibody mAb476 conjugated to enzyme alkaline phosphatase (AP) as the detection reagent, and finally placed in the mAB476 coated wells. Following further incubation, the bound Ab-Ag-Ab complex was detected via color development using a substrate of AP. It was observed that the samples with the known ligands or chelators had significantly greater antibody binding signal at low antigen concentrations compared to the untreated urine sample. This recapitulates the success of the methodology developed by the inventors to circumvent the IntL-1 competitive binding without extensive sample pre-treatment for detection of low antigen concentrations.

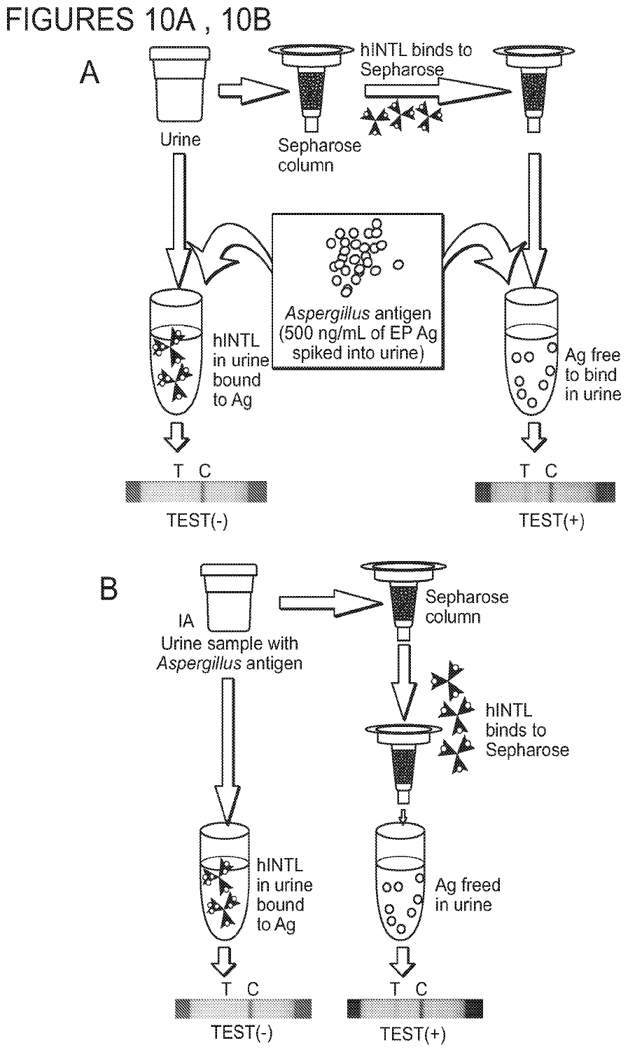

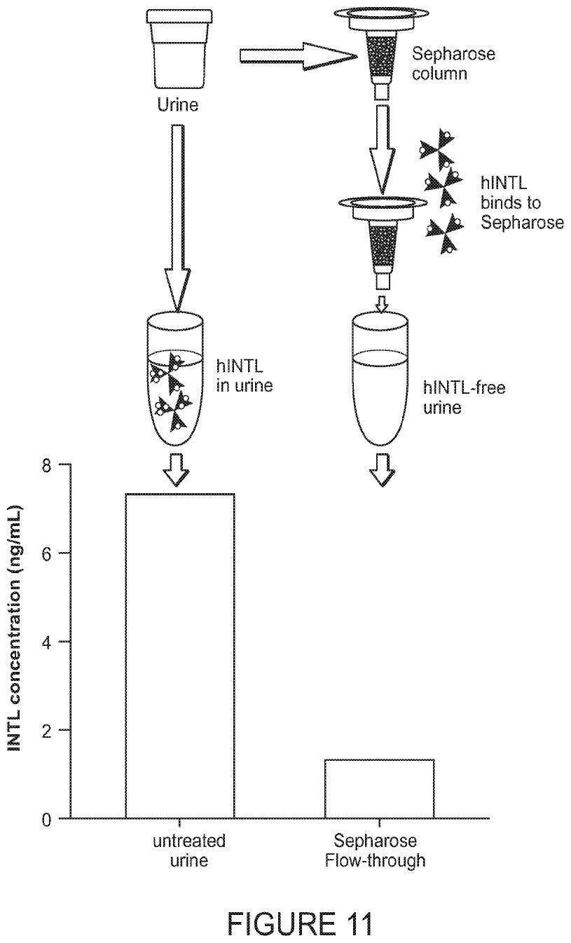

As shown in FIG. 10, intelectin is removed after passage of urine through sepharose. Sepharose slurry was placed in an empty column in a 100 microLiter volume. After removal of the holding buffer via a 1000×g spin for 2 minutes, urine (16 microLiter) was dropped on the sepharose, and incubated for 30 mins at room temperature. Flow-through was collected by another 1000×g spin for 2 minutes and intelectin was measured by ELISA.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for diagnosing a microbial infection in a biological sample from a mammalian subject suspected of having, having, or susceptible to having a microbial infection by detecting the presence of at least one polysaccharide comprising a galactofuranose residue in a biological sample of the mammalian subject, the method comprising:
(a) treating the biological sample to inhibit human intelectin-1 (hIntL-1) binding of galactofuranose residues present in the sample;
(b) contacting the treated sample of (a) with at least one antibody specific for at least one polysaccharide comprising a galactofuranose residue in an effective amount to produce a detectable amount of antibody-polysaccharide complex; and
(c) detecting the presence of at least one antibody-polysaccharide complex, wherein the detection of the presence of at least one antibody-polysaccharide complex is diagnostic of a microbial infection in a mammalian subject.

2. The method of claim 1, wherein in step (a) treating the sample comprises contacting the sample with a substrate which binds mono and divalent cations with high affinity.

3. The method of claim 1, wherein the substrate binds Ca²⁺ ions.

4. The method of claim 1, wherein in step (a) treating the sample comprises contacting the sample with a compound which chelates Ca²⁺ ions with high affinity.

5. The method of claim 1, wherein the compound is ethylenediaminetetraacetic acid (EDTA) and/or ethylene glycol-bis(β-aminoethyl ether)-N,N,N′,N′-tetraacetic acid (EGTA).

6. The method of claim 1, wherein in step (a) treating the sample comprises contacting the sample with a substrate which is bound by hIntL-1 with high affinity.

7. The method of claim 1, wherein the substrate is an antibody specific for hIntL-1.

8. The method of claim 1, wherein in step (a) treating the sample comprises contacting the sample with one or more compounds which are bound by hIntL-1 with high affinity.

9. The method of claim 1, wherein the one or more compounds, which are bound by hIntL with high affinity, comprise: glycerol, 3-Keto-2-deoxyoctonic acid (KDO), D-glycerol-1-phosphate, D-mannoheptose lactoferrin, saccharides, D-phospho-glycerol-modified glycans, heptoses, D-glycero-D-talo-oct-2-ulosonic acid (KO), 3-deoxy-D-manno-oct-2-ulosonic acid (KDO), sepharose or combinations thereof.

10. The method of claim 3, wherein the substrate comprises a desalting column.

11. The method of, wherein any one of claims 2 to 10 used in combination.

12. A method for diagnosing a microbial infection in a biological sample from a mammalian subject suspected of having, having, or susceptible to having a microbial infection, comprising:
(a) treating the biological sample to inhibit human intelectin-1 (hIntL-1) binding of galactofuranose residues present in the biological sample;
(b) contacting the treated sample of (a) with at least one antibody specific for at least one polysaccharide comprising a galactofuranose residue in an effective amount to produce a detectable amount of antibody-polysaccharide complex; and
(c) detecting the presence of at least one antibody-polysaccharide complex, wherein the detection of the presence of at least one antibody-polysaccharide complex is diagnostic of a microbial infection in a mammalian subject.

13. The method of claim 12, wherein in step (a) treating the sample comprises contacting the sample with one or more compounds, which are bound by hIntL with high affinity, comprising: glycerol, 3-Keto-2-deoxyoctonic acid (KDO), D-glycerol-1-phosphate, D-mannoheptose lactoferrin, saccharides, D-phospho-glycerol-modified glycans, heptoses, D-glycero-D-talo-oct-2-ulosonic acid (KO), 3-deoxy-D-manno-oct-2-ulosonic acid (KDO), sepharose, variants, derivatives or combinations thereof.

14. The method of claim 13, wherein the compound is sepharose or variants thereof.

15. The method of claim 12, wherein the sample is urine.

16. The method of claim 12, wherein the at least one antibody specific for at least one polysaccharide comprising a galactofuranose residue is selected from the group consisting of monoclonal antibody 205 (MAb 205) comprising a variable heavy (VH) domain of SEQ ID NO:1 and a variable light (VL) domain of SEQ ID NO:2; monoclonal antibody 24 (MAb 24) comprising a VH domain of SEQ ID NO:3 and a VL domain of SEQ ID NO:4; monoclonal antibody 686 (MAb 686) comprising a VH domain of SEQ ID NO:5 and a VL domain of SEQ ID NO:6; monoclonal antibody 838 (MAb 838) comprising a VH domain of SEQ ID NO:7 and a VL domain of SEQ ID NO:8; and monoclonal antibody 476 (MAb 476) comprising a VH domain of SEQ ID NO:9 and a VL domain of SEQ ID NO:10.

17. A method for diagnosing a microbial infection in a biological sample from a mammalian subject suspected of having, having, or susceptible to having a microbial infection, comprising:
(a) treating the biological sample with one or more compounds;
(b) contacting the treated biological sample of (a) with at least one antibody specific for a galactofuranose residue, in an effective amount to produce a detectable amount of antibody-polysaccharide complex; and
(c) detecting the presence of at least one antibody-polysaccharide complex, wherein the detection of the presence of at least one antibody-polysaccharide complex is diagnostic of a microbial infection in a mammalian subject; or
A method of detecting a microorganism in a sample, comprising:
(a) contacting the sample with one or more compounds comprising: glycerol, 3-Keto-2-deoxyoctonic acid (KDO), D-glycerol-1-phosphate, D-mannoheptose lactoferrin, saccharides, D-phospho-glycerol-modified glycans, heptoses, D-glycero-D-talo-oct-2-ulosonic acid (KO), 3-deoxy-D-manno-oct-2-ulosonic acid (KDO), sepharose, variants, derivatives or combinations thereof;
(b) contacting the sample form step (a) with an antibody which specifically binds to polysaccharides comprising galactofuranose;
(c) detecting the presence of at least one antibody-polysaccharide complex, thereby detecting the microorganism in the sample; or
A method of removing, eliminating, decreasing interference by human intelectin or decreasing the amount of human intelectin from a biological sample as compared to a control sample, comprising: contacting the biological sample containing the human intelectin with a one or more compounds comprising a linear carbohydrate or carbohydrate comprising an exocyclic diol under conditions permitting binding of the human intelectin to the linear carbohydrate; or
A support substrate or solution, comprising at least one antibody specific for at least one galactofuranose residue comprise: monoclonal antibody 205 (MAb 205) comprising a variable heavy (VH) domain of SEQ ID NO:1 and a variable light (VL) domain of SEQ ID NO:2; monoclonal antibody 24 (MAb 24) comprising a VH domain of SEQ ID NO:3 and a VL domain of SEQ ID NO:4; monoclonal antibody 686 (MAb 686) comprising a VH domain of SEQ ID NO:5 and a VL domain of SEQ ID NO:6; monoclonal antibody 838 (MAb 838) comprising a VH domain of SEQ ID NO:7 and a VL domain of SEQ ID NO:8; and monoclonal antibody 476 (MAb 476) comprising a VH domain of SEQ ID NO:9 and a VL domain of SEQ ID NO:10, or combinations thereof.

18. The method of claim 17, wherein the one or more compounds comprise: glycerol, 3-Keto-2-deoxyoctonic acid (KDO), D-glycerol-1-phosphate, D-mannoheptose lactoferrin, saccharides, D-phospho-glycerol-modified glycans, heptoses, D-glycero-D-talo-oct-2-ulosonic acid (KO), 3-deoxy-D-manno-oct-2-ulosonic acid (KDO), sepharose, variants, derivatives or combinations thereof.

19. The method of claim 18, wherein the compound is sepharose or variants thereof.

20. (canceled)

21. The method of claim 17, wherein the at least one antibody specific for at least one galactofuranose residue is selected from the group consisting of monoclonal antibody 205 (MAb 205) comprising a variable heavy (VH) domain of SEQ ID NO:1 and a variable light (VL) domain of SEQ ID NO:2; monoclonal antibody 24 (MAb 24) comprising a VH domain of SEQ ID NO:3 and a VL domain of SEQ ID NO:4; monoclonal antibody 686 (MAb 686) comprising a VH domain of SEQ ID NO:5 and a VL domain of SEQ ID NO:6; monoclonal antibody 838 (MAb 838) comprising a VH domain of SEQ ID NO:7 and a VL domain of SEQ ID NO:8; and monoclonal antibody 476 (MAb 476) comprising a VH domain of SEQ ID NO:9 and a VL domain of SEQ ID NO:10.

22-31. (canceled)


SEQUENCE LISTING (10 sequences)

SEQ ID NO: 1 - DNA, 339 bp, Mus musculus
gaggtgcagctggaggagtctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcatcgtctccagagacacttcccaaagcatcctctaccttcagatgaatgccctgagagctgaggacactgccatttattactgtgcaagagattactacggtagtagctactggtacttcgatgtctggggcgcagggaccacggtcaccgtctcctca

SEQ ID NO: 2 - DNA, 341 bp, Mus musculus
gacattctgatgacccagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagctatcctctcacgttcggctcggggacaaagttggaaataaaacg

SEQ ID NO: 3 - DNA, 359 bp, Mus musculus
  misc_feature (18)..(18): n is a, c, g, or t
gaggtgcagctggaggantctgggggagacttagtgaagcctggagggtccctgaaactctcctgtgcagcctctggattcactttcagtagctatggcatgtcttgggttcgccagactccagacaagaggctggagtgggtcgcaaccattagtagtggtggtagttacacctactatccagacagtgtgaaggggcgattcaccatctccagagacaatgccaagaacaccctgtacctgcaaatgagcagtctgaagtctgaggacacagccatgtattactgtgcaagacttactacggtagtagctatgctatggactactggggtcaaggaacctcagtcaccgtctcctca

SEQ ID NO: 4 - DNA, 341 bp, Mus musculus
gacattctgatgacccagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagctatcctctcacgttcggtgctgggaccaagctggagctgaaacg

SEQ ID NO: 5 - DNA, 341 bp, Mus musculus; identical to SEQ ID NO: 4

SEQ ID NO: 6 - DNA, 341 bp, Mus musculus
gacattctgatgacccagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagctatccgctcacgttcggtgctgggaccaagctggagctgaaacg

SEQ ID NO: 7 - DNA, 342 bp, Mus musculus
gaggtgcagctggaggagtctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcttcgtctccagagacacttcccaaagcatcctctaccttcagatgaatgccctgagagctgaggacactgccatttattactgtgcaagagatgttatgattacgacgggggactggtacttcgatgtctggggcgcagggaccacggtcaccgtctcctca

SEQ ID NO: 8 - DNA, 318 bp, Mus musculus
gacattctgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccagtacagtggagtccctgatcgcttcacaggcagtggatctgggacagatttcactctcaccatcagcaatgtgcagtctgaagacttggcagagtatttctgtcagcaatttaacagctatcacgttcggctcggggacaaagttggaattaaaacg

SEQ ID NO: 9 - DNA, 375 bp, Mus musculus
gaggtgcagctggaggagtctggaggaggcttggtacagcctgggggttctctgagactctcctgtgcaacttctgggttcaccttcagtgatttctacatggagtgggtccgccagactccaggggagagactggagtggattgctgcaagtagaaacaaagctaatgattatacagcagaatacagtgcgtctgtgaagggtcgattcaccgtctttagagacacttcccaaaacatcctctaccttcagatgaatgccctgagagctgaagacactgccgcctattactgtgcaagagatgcggactacggtaaaaccttttcctggtacttcgatgtctggggcgcagggaccacggtcaccgtctcatca

SEQ ID NO: 10 - DNA, 338 bp, Mus musculus
gatattgtaatgacccaagatgaactctccaatcctgtcacttctggagaatcagttcacatctcctgcaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtagagtgaaggctgaggatgtgggtgtgtattattgtcaacaacttgtagaatatccgctcacgttcggtgctgggaccaagctggagctgaaacg